(19) United States
(12) Patent Application Publication
(10) Pub. No.: US 2012/0003664 A1
(43) Pub. Date: Jan. 5, 2012

(54) METHOD FOR EVALUATING PRE-TREATMENT

(76) Inventors: Uffe Lovborg, Tuscon, AZ (US); Zhiming Liao, Ventura, CA (US); Clive R. Taylor, Talibu, CA (US)

(21) Appl. No.: 13/133,558

(22) PCT Filed: Dec. 9, 2009
(86) PCT No.: PCT/DK09/00249

§ 371 Date: Sep. 23, 2011

Related U.S. Application Data
(60) Provisional application No. 61/120,930, filed on Dec. 9, 2008.

(30) Foreign Application Priority Data
Dec. 15, 2008 (DK) PA 208 01 784

(51) Int. Cl. G01N 33/53

(52) U.S. Cl. 435/7.1

(57) ABSTRACT

The present invention relates to methods for evaluating tissue pre-treatment such as ischemic time, fixation time and alcohol time in an immunohistochemical assay by using one or more internal controls. Said internal controls may be biomarker specific or tissue specific. Also included are uses and kits comprising said internal controls.

### FIELD OF INVENTION

The present invention relates to a method for evaluating tissue pre-treatment, comprising including in an immunohistochemical process an internal control comprising one or more antibody that demonstrates variations in accessibility or binding capacity relative to variations in tissue pre-treatment.

### BACKGROUND OF THE INVENTION

Recommendations for pre-treatment of patient tissues prior to immunohistochemical (IHC) testing exist, but these may vary and may or may not be adhered to. By example, the time it takes from resection of tissue from a patient until it is placed in formalin may vary. This is also called ischemic time. Another variation is the formalin time, e.g. when shipping patient tissue in a formalin container from surgery to test lab, the tissue may be in transit for varying length of time and when tissues are treated in a processor, the programmed time for formalin fixation may be different from lab to lab.

With many variation and possible little knowledge of these variations, the pre-analytical parameters may vary in ways that could adversely affect the IHC signal.

Recommendations for proper formalin fixation range from 6 to 48 hours, preferably 12-24 hours. This time frame is often, but not always followed e.g. if the time frame does not fit within the normal work-schedule. An example could be a sample that is put in formalin on Friday morning. A lab with normal working hours can then chose either to do a short fixation and process the sample in the afternoon or leave the sample in formalin till Monday morning. This everyday scenario leads to great variation in fixation between samples.

Formalin actually fixes in two steps. The first step is the penetration of the fixative into the tissue; effectively stopping the metabolic processes. This process is in the range of 1 mm/hour and thus may take hours to complete. The general recommendation is therefore to cut tissues in small pieces to facilitate faster penetration, but this is not always adhered to. The second step is even slower wherefore long formalin incubation time is often used. It consists of the formation of a more stable bond, or “crosslinking”, between the remaining reactive end of the formaldehyde molecule onto the same macromolecule with the loose bond or with one that is nearby. This causes firming of the macromolecules and masking of biologically active sites.

Different tissue types have different rates of reactivity during fixation. During fixation tissue proteins are changed and or cross linked and the epitope (i.e. the part that antibodies would react and bind to in the protein or antigen) may be hidden or masked and therefore becomes unavailable to the antibody. Due to the epitope placement in the proteins 3 dimensional structure some epitopes are masked within minutes while others can take days or longer to show signs of weakening.

Any changes caused by formalin fixation create the microscopic morphologic images that are familiar to pathologists and that have been the basis for pathologic diagnosis for many years. Changes outside the norm or changes to epitope availability but with unimpaired morphology will not be observable to the pathologist microscopically.

The variations to pre-analytical parameters of the pre-treatment include, but not limited to:

A. Ischemic time, i.e. time from resection or removal of tissue from a patient to start of formalin fixation. When a tissue is removed from a living body it will lose both the supply of oxygen and nutrients and the removal of waste products. While the life processes slowly stop, the tissue will start degrading with loss of protein structure and function and of morphology. The production of some proteins may also be up-regulated as a reaction to the changes occurring in the tissue.

The time before start of formalin fixation is therefore a factor that influences the subsequent immunohistochemical process. Both an increase and a decrease in staining may be the effect of extended ischemic time depending on the protein.

B: Formalin time, i.e. time in formalin. When a tissue is immersed in formalin the life processes are “fixed in time”. Therefore all degradation will stop and the tissue will be preserved as it was at the start of formalin action. Formalin penetration into the center of the tissue takes time, but tissue pieces of appropriate size will be penetrated by formalin before any adverse effects will happen and the entire tissue piece will be suitable for analysis. The proteins will be chemically cross-linked to other cellular components thus preventing that they are washed out of the tissue during the analytical steps. Appropriately formalin fixed tissue will therefore have intact morphology and protein structure, function and recognizability. The IHC test of this tissue will result in correct response or signal.

Too short a time in formalin may occur. If the time in formalin is too short the tissue will not be well preserved and will experience degradation with impaired morphology and protein integrity. Similar effects may occur in the center of tissue pieces, especially large pieces. As formalin takes time to diffuse into a tissue, the centermost parts of a tissue will be exposed to formalin for shorter time than the rim. Centers of large pieces of tissues may therefore be less preserved than the rim, with corresponding loss of protein integrity and or tissue morphology. This may be observed as loss of IHC signal and deteriorating morphology. Alternatively, an increased IHC signal may be observed due to the absence of the chemical cross-links that partially restrict access by the antibodies to their targets. Sufficient time is needed for the formalin to penetrate into the center of the tissue piece. Even then the center may have suffered changes already and it is usually recommended to segment the tissue into smaller pieces before processing.

Too long time in formalin may occur. As the action of formalin includes cross linking of protein and this increases with time, proteins in tissues exposed to formalin for long time versus short time will be more cross linked, with corresponding risk for loss of accessibility of antibody. This may be observed as loss of IHC signal. However, the morphology may not be impaired.

In conclusion, the time in formalin is a factor that will affect the intensity of the IHC signal, both if it is too long and too short. The effect may be both towards a higher and a lower intensity depending on the protein.

C. Alcohol time

Alcohol has two main effects on tissues. One is dehydration of the tissue thereby preparing it for later paraffin infiltration the other is fixation of in particular proteins.

In preparation of the paraffin infiltration of the tissue, residual formalin and all water must be removed as paraffin and water doesn't mix at regular temperatures. The water is removed in a series of exposures to increasingly concentrated alcohols. Often a sequence of 70%-95%-100%-100% is used where the last will remove the final amount of water from the tissue. After exposure to the organic solvent xylene the tissues are placed in warm molten paraffin, which will fill up all spaces originally taken up by water and fat. The end result is a tissue that is filled with paraffin and that may be embedded into a paraffin block for sectioning, where sections are placed on microscopy slides for test. The de-hydration effect of alcohol is therefore a step on the way to making a block of tissue.

Where formalin fixation is applied the fixation effect of alcohol is of lesser importance as the effect of formalin is so strong. Alcohols fixing effect may anyhow become relevant where insufficient/in-complete formalin fixation has been done. Using tissue processing protocols with hour long alcohol incubation may therefore minimize the effect of formalin underfixation and any generalized underfixation effects may be seen less frequently that could be expected. There are however also proteins that are reported to be sensitive to alcohol fixation. This could be either because the lack of cross-linking allows the protein to be washed out of the tissue during analysis, or that the precipitative action of the alcohol fixation changes the conformation of the protein thereby disrupting the epitope recognized by the antibody.

Alcohol fixation may thus compensate for insufficient formalin fixation and thus preserve the tissue and lead to correct IHC-results. In few cases, there may however be a negative effect of alcohol fixation leading to reduced IHC signal. The effect is dependent on the protein being analysed.

There is thus a need to provide a method for determining pre-analytical parameters, particularly if optimum pre-analytical guidelines have been upheld such that the subsequent immunohistochemical process will provide accurate results in a simple and reliable way, as well as to provide an internal control in the same sample as an analysis is being performed.

Accordingly, the present invention seeks to provide means and methods to perform accurate and less biased IHC assays, such as IHC based diagnostic assays, in a simple and efficient way for routine testing when diagnosing or prognosing pathological conditions.

### SUMMARY OF THE INVENTION

One aspect of the present invention provides a method for evaluating tissue pre-treatment in an immunohistochemical process, comprising
a) providing a formalin fixed biological sample,
b) providing an internal control comprising one or more antibody that demonstrates variations in antigen accessibility or variations in antibody binding capacity to said antigen relative to variations in tissue pre-treatment
c) detecting said variations in antigen accessibility or variations in antibody binding capacity to said antigen in step b), and
d) analyzing said variations detected in step c) relative to variations in tissue pre-treatment thereby evaluating tissue pre-treatment.

Further embodiments are wherein the variation in tissue pre-treatment comprises fixation time, ischemic time and alcohol time.

Further embodiments are wherein the variation in tissue pre-treatment is fixation time.

Further embodiments are wherein the variation in tissue pre-treatment is ischemic time.

Further embodiments are wherein the variation in tissue pre-treatment is alcohol time.

Further embodiments are wherein one or more antibody is 1, 2, 3, 4, 5, 6, 7, 8, 9, 10, 11, 12, 13, 14, 15, 16, 17, 18, 19, 20, or even more antibodies.

Further embodiments are wherein the method further comprises providing at least one or more analyzing antibody. The analyzing antibody may also be a clinical antibody useful in diagnosis of a pathological condition. The pathological condition may be any pathological condition. In one embodiment, the pathological condition is cancer. Examples of analytical, or analyzing, antibodies are antibodies binding specifically to cKit, Her2, EGFR. Further examples are antibodies binding specifically to Hepatocyte antigen, BRCA1, and melanoma.

Even further embodiments are wherein the one or more antibody that demonstrates variations in antigen accessibility or variations in antibody binding capacity to said antigen is selected from the list consisting of antibodies binding specifically to CD3, S100, Melan A, Villin, ER α (Estrogen Receptor alpha), CD20, EMA, E-Cadherin, CD9, Vimentin, IgG, Kappa, Myeloperoxidase, CD18, 34βE12, Chromogranin A, Mammaglobin, CD31, Caldesmon, CD15, CK19 and 34βE12.

Even further embodiments are wherein the variation in tissue pre-treatment is fixation time and the one or more antibody that demonstrates variations in antigen accessibility or variations in antibody binding capacity to said antigen is selected from the list consisting of antibodies binding to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15, CK19 and 34βE12.

Still even further embodiments are wherein the variation in tissue pre-treatment is ischemic time and the one or more antibody that demonstrates variations in antigen accessibility or variations in antibody binding capacity to said antigen is selected from antibodies binding to phosphorylated antigens.

In one embodiment the phosphorylated antigen is pAkt.

Further embodiments are wherein the variation in tissue pre-treatment is alcohol time and the one or more antibody that demonstrates variations in antigen accessibility or variations in antibody binding capacity to said antigen is antibodies binding to Estrogen Receptor (ER) α.

Further embodiments are wherein the method further comprises determining if said tissue pre-treatment is acceptable based on the detected and analyzed variations.

Further embodiments are wherein the internal control is tissue specific.

Further embodiments are wherein the internal control is specific for a tissue. Examples of tissues are stomach, small intestine, colon, liver, kidney, heart, lung, duodenum, tongue, pylorus, pancreas, uterus, skin, gal bladder, urinary bladder, adrenal, muscle, and ovary. The tissue specific control is always uniformly expressed in these tissues and respond to variations in tissue-pretreatment in a reproducible.

In one embodiment the tissues are selected from the list consisting of stomach, small intestine, colon, liver, kidney, heart, lung, duodenum, tongue, pylorus, pancreas, uterus, skin, gal bladder, urinary bladder, adrenal, muscle, and ovary.

Further embodiments are wherein the tissue is human tissue.

Further embodiments are wherein the internal control is biomarker specific. A biomarker may be a protein (as in IHC), DNA, RNA or a metabolic compound, a carbohydrate. The biomarker may also be a clinical marker. Examples are given herein.

Further embodiments are wherein the biomarker specific control is an antibody that recognizes a second marker that displays the same sensitivity as the biomarker to variations in tissue-pretreatment.

Further objects of the present invention is the use of an internal control for determining tissue pre-treatment variations in an immunohistochemical process.

Further embodiments are wherein the internal control comprises one or more antibodies binding specifically to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15 or CK19 as an internal control determining tissue pre-treatment variations in an immunohistochemical process.

Further embodiments are wherein the internal control is tissue specific.

Further embodiments are wherein the internal control is biomarker specific.

### DETAILED DESCRIPTION OF THE INVENTION

### Definitions

“Subject” as used herein, means any mammal including human having or suspected of having a disease.

“At least one” or “one and more” as used herein means one or more, i.e. 1, 2, 3, 4, 5, 6, 7, 8, 9, etc., or as else is suggested herein depending on the context.

“Detection”, “detect”, “detecting” as used herein includes qualitative and/or quantitative detection (measuring levels) with or without reference to a control, and further refers to the identification of the presence, absence, or quantity of a given protein.

As used herein, the singular forms “a”, “and”, and “the” include plural referents unless the context clearly dictates otherwise. Thus, for example, reference to “an antibody” includes a plurality of such antibodies.

“Immunohistochemistry” (IHC) is in the present context to be understood to include all presentation form of tissue on a substrate, such a glass slide, including tissue microarrays or any other means disclosed herein.

As used herein “pre-treatment” of tissue is intended to mean any pre-treatment that may affect the tissue during the time frame from resection of a tissue up till and including the preparation of the tissue before storage until the analytical process steps of an immunohistochemical assay takes place. Thus, said pre-treatment may influence the tissue morphology including protein structure and function. Examples of tissue pre-treatment are given herein and include ischemic time i.e. ischemic pre-treatment, formalin fixation i.e. formalin pre-treatmemt, and alcohol time i.e. alcohol pre-treatment.

Is should be noted that the ischemic time is a time frame from resection or removal of the tissue till start of the fixation, normally a formalin fixation. During this time frame said tissue is affected due to lack of or slow fixation process, the latter due to thickness of the tissue, thereby influencing the tissue morphology and the protein structure and function. The lack of fixation simply starts tissue degradation. Thus, ischemic time, or ischemic pre-treatment, is included in said tissue-pretreatment since it is affecting the tissue simply by lack of fixation.

In the context of the present invention ‘fixation time’ is to be understood as the time from when the biological samples is affected by fixation medium until when it is not affected any more. This could for example be the time from immersion into the fixation medium until the time the biological sample is removed from the fixation medium.

In the context of the present invention ‘ischemic time’ is to be understood as the time from removal of biological sample from a biological system, such as an animal or human, until said biological samples gets into contact with the fixation medium.

In the context of the present invention ‘alcohol time’ is to be understood as the time a tissue is present in graded or absolute alcohol after fixation in formalin. This is some times also referred to as the dehydration step during tissue processing and will often be a sequence of increasing percentage of alcohols, often starting at 70% and continuing with 95%, 100% and 100% where the last will remove the final low amount of water that would have been present.

As used herein, “pre-analytical conditions” are intended to mean conditions during the tissue pre-treatment.

As used herein, “pre-analytical parameters” are intended to mean a set of measurable factors such as e.g. time and temperature, that define a system, here pre-analytical treatment or pre-treatment, and that determine its, i.e. the systems, condition and that such parameters may be varied. The pre-analytical parameters may thereby create a variability in the pre-analytical condition(s). Said variability affects the tissue, such as morphology and protein structure and integrity, thereby affecting the subsequent analytical IHC assay and its result(s).

In the present context of the invention ‘variations in accessibility or binding capacity relative to variations in tissue pre-treatment’ means variations in IHC signal relative to variation in pre-analytical conditions. For example, an epitope can be fully or partially masked during fixation such that antibody binding is decreased in the subsequent immunohistochemical process.

As used herein, “accessibility” is intended to refer to the accessibility of a protein by an antibody, or more precisely, to the accessibility of an antigen to which an antibody binds specifically.

As used herein, “binding capacity” is intended to refer to the binding capacity or binding capability of an antibody to a protein, or more precisely, an antigen to which said antibody binds specifically to.

As used herein, a “biomarker” is a substance in a tissue or biological fluid that is used as an indicator of a biological state. A biological state is the result of a normal biological process or a pathological process. The substance may be, but not limited to, proteins, carbohydrates, DNA, RNA, hormones or metabolic compounds. The presence of the biomarker will change as the process, normal or pathological, changes thus affecting the biological state.

As used herein, a “tissue-marker” is a substance in a tissue or a biological fluid that is always present in a particular tissue or organ or cell-type irrespective of the state that tissue or organ or cell-type is in as opposed to a biomarker that is an indicator of a biological state or process. Certain proteins may be tissue-markers. However, the same tissue-marker may also be used as a biomarker of e.g. a pathological state if found in another tissue or organ or cell-type

“Diagnosis” as used herein encompasses the identification of the nature of a disease.

“Prognosis” as used herein encompasses a forecast as to the probable outcome of a disease, the prospects as to recovery from a disease as indicated by the nature and symptoms of a disease.

“Subject” as used herein denotes a mammal, such as a rodent, a feline, a canine, and a primate. Preferably a subject according to the invention is a human.

“Monoclonal antibody” or “mAb” as used herein refers to an antibody of a single amino acid composition, that is directed against a specific antigen and that is produced by a single clone of B cells or hybridoma.

“Polyclonal antibody” as used herein refers to an antibody that is directed against a specific antigen that is derived from different B-cell lines.

“Fab” as used herein refers to an antibody fragment having a molecular weight of about 50,000 Da and antigen binding activity, in which about a half of the N-terminal side of H chain and the entire L chain, among fragments obtained by treating IgG with a protease, papaine, are bound together through a disulfide bond.

“F(ab′)₂” as used herein refers to an antibody fragment having a molecular weight of about 100,000 Da and antigen binding activity, which is slightly larger than the Fab bound via a disulfide bond of the hinge region, among fragments obtained by treating IgG with a protease, pepsin.

“Fab′” as used herein refers to an antibody fragment having a molecular weight of about 50,000 Da and antigen binding activity, which is obtained by cutting a disulfide bond of the hinge region of the F(ab′)₂.

As used herein, a single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, preferably by using gene recombination techniques.

“Hybridoma” as used herein denotes a cell, which is obtained by subjecting a B cell prepared by immunizing a non-human mammal with an antigen to cell fusion with a myeloma cell derived from a mouse or the like which produces a desired monoclonal antibody having an antigen specificity.

As used herein a “biological sample” encompasses a variety of sample types obtained from any subject. A typical subject is a human. Exemplary biological samples useful in the disclosed methods include but are not limited to biological samples disclosed herein such as e.g. solid tissue samples such as a biopsy specimen or tissue cultures or cells derived there from, and the progeny thereof. For example, biological samples include cells obtained from a tissue sample collected from an individual. Therefore, biological samples encompass clinical samples, cells in culture, cell supernatants, cell lysates, and tissue samples, e.g. a biopsy.

Samples may be fresh or processed post-collection (e.g., for archiving purposes). In some examples, processed samples may be fixed (e.g., formalin-fixed) and/or wax- (e.g., paraffin-) embedded. Fixatives for mounted cell and tissue preparations are well known in the art and include, without limitation, 95% alcoholic Bouin's fixative; 95% alcohol fixative; B5 fixative, Bouin's fixative, formalin fixative, Karnovsky's fixative (glutaraldehyde), PLP (Periodate/Lysine/Paraformaldehyde), paraformaldehyde, Boonfix I, Boonfix II, Myrsky fixative, glutaraldehyde, zinc formalins Karnovsky's fixative (glutaraldehyde), Hartman's fixative, Hollande's fixative, Orth's solution (dichromate fixative), and Zenker's fixative or other aldehydes or other bi-functional cross-linkers (see, e.g., Carson, Histotechology: A Self-Instructional Text, Chicago:ASCP Press, 1997). In some examples, the sample (or a fraction thereof) is present on a solid support.

Cytological preparations may be fixed using e.g. acetone, acetone/methanol, acetone/methanol/formaldehyde, formaldehyde, Zambonis fixative (paraformaldehyde and picrine acid).

Solid supports useful in a disclosed method need only bear the biological sample and, optionally, but advantageously, permit convenient detection of the proteins of interest in the sample. Exemplary supports include microscope slides (e.g., glass microscope slides or plastic microscope slides), coverslips (e.g., glass coverslips or plastic coverslips), tissue culture dishes, multi-well plates, membranes (e.g., nitrocellulose or polyvinylidene fluoride (PVDF)) or BIACORE®; chips.

The term “algorithm” as used herein refers to a mathematical formula that provides a relationship between two or more quantities. Such a formula may be linear, or non-linear, and may exist as various numerical weighting factors in computer memory.

### Methods of the Invention, Antibodies and Scoring

Until now it has not been possible to determine if a specific immunohistochemical (IHC) test, such as a diagnostic assay, conducted on a tissue will be correct or not due to variations in pre-analytical parameters. If steps taken during fixation vary considerably from the optimum this can have detrimental effect of the analysis of the sample, and even lead to incorrect diagnosis.

The present invention counteracts the lack of standardization and lack of knowledge of the treatment of individual tissues and enables testing of the pre-analytical parameters and provides guidance for which changes may have impact on the analysis. With the present invention it will be possible to realize if a specimen pre-treatment was correct with respect to the following immunohistochemical process, and, hence, whether results from a specific IHC test conducted on said tissue will be correct or not. Finally, guidance can be given to describe measures to obtain a correct result.

In the methods and uses disclosed where protein expression is determined by immunohistochemistry a scoring of protein expression may optionally be used. The scoring may be semi-quantitative; for example, with protein expression levels recorded as 0, 1, 2, 3 or 4 (including, in some instances plus (or minus) values at each level, e.g., 1+, 2+, 3+, 4+) with 0 being substantially no detectable protein expression and 4 (or 4+) being the highest detected protein expression. In such methods, an increase or decrease in the corresponding protein expression is measured as a difference in the score as compared the applicable control (e.g. a standard value or a control sample); that is, a score of 4+ in a test sample as compared to a score of 0 for the control represents increased protein expression in the test sample, and a score of 0 in a test sample as compared to a score of 4+ for the control represents decreased protein expression in the test sample.

In IHC antibodies (e.g., monoclonal and/or polyclonal antibodies) specific for each protein are used to detect said protein. The antibodies can be detected, as further described herein, by direct labelling of the antibodies themselves, for example, with radioactive labels, fluorescent labels, hapten labels such as, biotin, or an enzyme such as horseradish peroxidase or alkaline phosphatase. Alternatively, an indirect labelling is used where unlabeled primary antibody is used in conjunction with a labelled secondary antibody, comprising e.g. antiserum, polyclonal antiserum or a monoclonal antibody specific for the primary antibody. IHC protocols are well known in the art and are commercially available, see e.g. Antibodies: A Laboratory Manual, Harlow and Lane (Cold Spring Harbor Laboratory press, Cold Spring Harbor, N.Y. 1988) and Current Protocols in Immunology, and Current Protocols in Molecular Biology, both John Wiley and Sons, Inc., N.Y.) incorporated herein by reference.

By “reacting specifically with” as used herein it is intended to equal “capable of binding selectively” or “binding specifically to”. As used herein the expressions are intended to mean that the antibody or antigen-binding fragment, or variant, fusion or derivative thereof, including any anti-body derived binding moiety, which is capable of binding to an antigen of a molecule and further which binds at least 10-fold more strongly the proteins. than to another proteins for example at least 50-fold more strongly or at least 100-fold more strongly. The binding moiety may be capable of binding selectively to the protein under physiological conditions, e.g. in vivo. Suitable methods for measuring relative binding strengths include, immunoassays, for example where the binding moiety is an antibody (see Harlow & Lamp; Lane, “Antibodies: A Laboratory”, Cold Spring Harbor Laboratory Press, New York, which is incorporated herein by reference). Alternatively, binding may be assessed using competitive assays or using Biacore® analysis (Biacore International AB, Sweden).

The antibodies may in further aspects of the present invention be provided in an antibody cocktail, in aqueous form or in a freeze dried powder form. For the latter, a re-hydration step is required to put the antibodies in a usable liquid form before use. The antibodies may be provided in a concentrated form or in a ready-to-use form.

The antibodies may be whole antibodies or fragments thereof, e.g. antigen-binding fragment, or variant, fusion or derivative thereof as long as they are capable of binding to the desired protein in vitro. Such binding specificity may be determined by methods well known in the art, such as e.g. ELISA, immunohistochemistry, immunoprecipitation, Western blots, chromatography and flow cytometry using transfected cells expressing the all subunit or a heterodimer thereof (see Examples). Examples of how to measure specificity of an antibody is given in e.g. Harlow & Lane, “Antibodies: A Laboratory”, Cold Spring Harbor Laboratory Press, New York, which is incorporated herein by reference.

By “antibody” we include substantially intact antibody molecules of any species such as rodents, e.g. murine, rat, guinea pig, or non-rodents such as rabbit, goat, sheep, dog, pig, camel, dromedary, donkey, horse or chicken, as well as chimeric antibodies, humanized antibodies, human antibodies (wherein at least one amino acid is mutated relative to the naturally occurring human antibodies), single chain antibodies, bispecific antibodies, antibody heavy chains, antibody light chains, homo-dimers and hetero-dimers of antibody heavy and/or light chains, and antigen binding fragments and derivatives of the same. For example, the antibody may be a monoclonal antibody.

Antigenic specificity is conferred by variable domains and is independent of the constant domains, as known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V H and V L partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

Thus, by “antigen-binding fragment” we mean a functional fragment of an antibody that is capable of binding specifically to a protein.

Exemplary antigen-binding fragments may be selected from the group consisting of Fv fragments (e.g. single chain Fv and disulphide-bonded Fv), Fab-like fragments (e.g. Fab fragments, Fab′ fragments and F(ab) 2 fragments), single antibody chains (e.g. heavy or light chains), single variable domains (e.g. VH and VL domains) and domain antibodies (dAbs, including single and dual formats; i.e. dAb-linker-dAb).

Thus, in one embodiment the antibody or antigen-binding fragment, or variant, fusion or derivative thereof, comprises or consists of an intact antibody. In one embodiment, the antibody is a monoclonal antibody.

For example, the antibody or antigen-binding fragment, or a variant, fusion or derivative thereof, may consist essentially of an intact antibody. By “consist essentially of we mean that the antibody or antigen-binding fragment, variant, fusion or derivative thereof consists of a portion of an intact antibody sufficient to retain binding specificity to a protein.

In further embodiments, the protein is of human origin.

The term ‘antibody’ also includes all classes of antibodies, including IgG, IgA, IgM, IgD and IgE. In one embodiment, however, the antibody is an IgG molecule, such as an IgGl, IgGl, IgG3, or IgG4 molecule.

In one embodiment, the antibody is an IgG1 molecule. In a further embodiment, the antibody is a IgG1 molecule with a kappa light chain.

In a further embodiment, the antibody is a non-naturally occurring antibody. Of course, where the antibody is a naturally occurring antibody, it is provided in an isolated form (i.e. distinct from that in which it is found in nature).

Also included within the scope of the invention are modified versions of antibodies and antigen-binding fragments thereof, e.g. modified by the covalent attachment of polyethylene glycol or other suitable polymer, and uses of the same.

Methods of generating antibodies and antibody fragments are well known in the art. For example, antibodies may be generated via any one of several methods which employ induction of in vivo production of antibody molecules, screening of immunoglobulin libraries (Orlandi. et al, 1989. Proc. Natl. Acad. Sci. U.S.A., vol 86, pages 3833-3837; Winter et al, 1991, Nature 349:293-299, which are incorporated herein by reference) or generation of monoclonal antibody molecules by cell lines in culture. These include, but are not limited to, the hybridoma technology, the human B-cell hybridoma technology, and the Epstein-Barr virus (EBV)-hybridoma technology (see Kohler et al, 1975. Nature 256:4950497; Kozbor et al, 1985. J. Immunol. Methods 81:31-42; Cote et al, 1983. Proc. Natl. Acad. Sci., USA 80:2026-2030; Cole et al, 1984. Mol. Cell. Biol. 62:109-120, which are incorporated herein by reference).

For example, generating monoclonal or poloclonal antibodies to a protein may be done by immunization where the whole protein or a suitable fragment thereof can be injected into non-human mammals (such as mice or rabbits), followed by boost injections, to produce an antibody response. Serum isolated from immunized animals may be isolated for the polyclonal antibodies contained therein, or spleens from immunized animals may be used for the production of hybridomas and monoclonal antibodies.

In one example, a monoclonal antibody to one of the proteins can be prepared from murine hybridomas according to the classical method of Kohler and Milstein {Nature, 256:495, 1975) or derivative methods thereof. Briefly, a mouse (such as Balb/c) is repetitively inoculated with a few micrograms of the selected protein or peptide fragment thereof or a suitable carrier conjugate thereof over a period of a few weeks. The mouse is then sacrificed, and the antibody-producing cells of the spleen isolated. The spleen cells are fused by means of polyethylene glycol with mouse myeloma cells, and the excess un-fused cells destroyed by growth of the system on selective media comprising aminopterin (HAT media). The successfully fused cells are diluted and aliquots of the dilution placed in wells of a microtiter plate where growth of the culture is continued.

Antibody-producing clones are identified by detection of antibody in the supernatant fluid of the wells by immunoassay procedures, such as ELISA, as originally described by Engvall (Enzymol., 70:419, 1980), and derivative methods thereof.

Selected positive clones can be expanded and their monoclonal antibody product harvested for use.

Commercial sources of antibodies include DAKO A/S, Abcam, Lab Vision, BioCare Medical, Cell Marque Corp., etc.

Polyclonal antibody-producing animals are identified by bleeding immunised animals and selection of appropriate animal with ha suitable polyclonal antibody-titer thereof.

In some embodiments, antibodies are purified before use. Purification of antibodies are done using techniques available in the art and described in e.g. “Monoclonal Antibodies: A manual of techniques”, H Zola (CRC Press, 1988) and in “Monoclonal Hybridoma Antibodies: Techniques and Applications”, J G R Hurrell (CRC Press, 1982), which are incorporated herein by reference.

Generation of antibodies proteins mentioned herein are described in the art and available from commercial sources as described herein, or being available using techniques known to a skilled artisan using references enclosed herein and accordingly incorporated herein by reference.

The antibody or antigen-binding fragment or derivative thereof may also be produced by recombinant means. Suitable monoclonal antibodies to selected antigens and proteins may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982), and “Antibodies: A Laboratory Manual”, Cold Spring Harbor Laboratory, New York, which are incorporated herein by reference.

Antibody fragments can also be obtained using methods well known in the art (see, for example, Harlow & Lane, 1988, “Antibodies: A Laboratory Manual”, Cold Spring Harbor Laboratory, New York, which is incorporated herein by reference). For example, antibody fragments may be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

Further provided herein is that the primary antibodies or fragments thereof may be labelled directly or indirectly, with a detectable moiety. By directly labelled is meant that the detectable moiety is attached to the antibody. By indirect labelled it is meant that the detectable moiety is attached to a linker, such as, for example, a secondary or tertiary antibody. The detectable moiety may be any moiety or marker known to those skilled in the art, or as described herein, and as being such a moiety being capable of generating a signal that allows the direct or indirect qualitative or quantitative or relative measurement of a molecule to which it is attached.

A wide variety of detectable moieties, or labels, and conjugation techniques are known and reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149; 4,366,149 and 4,366,241 (all incorporated herein by reference). Also, recombinant immunoglobulins may be used and labelled (see U.S. Pat. No. 4,816,576, incorporated herein by reference).

The detectable moiety may be a single atom or molecule which is either directly or indirectly involved in the production of a detectable species. Optionally, the detectable moiety is selected from the group consisting of a fluorescent moiety, an enzyme linked moiety, a biotinylated moiety and a radiolabeled moiety, as described further herein, e.g. below. By “label”, “detectable moiety” is meant any detectable tag that can be attached directly (e.g., a fluorescent molecule integrated into a polypeptide) or indirectly (e.g., by way of binding to a primary antibody with a secondary, tertiary or further antibody with an integrated fluorescent molecule) to the molecule of interest. Thus, a label, marker or detectable moiety is any tag that can be visualized, for example, with imaging methods.

By a “detectable moiety” we further include the meaning that the moiety is one which, when located at the target site following providing an antibody to a biological sample, such as a tissue sample, e.g. a human tissue sample, may be detected in vitro. That includes that the detectable moiety is signal generating and it is further convenient and thus included in further embodiments if the detectable moiety may be detected and the relative amount and/or location of the moiety (for example, the location on an tissue sample) may be determined. Detectable moieties are well known in the art and is included in different detection systems.

Suitable detectable moieties are well known in the art and the attachment or linking of these moieties to polypeptides and proteins is further well known in the art. Further examples of detectable moieties are an enzyme; an enzyme substrate; an enzyme inhibitor; coenzyme; enzyme precursor; apoenzyme; fluorescent substance; pigment; chemiluminescent compound; luminescent substance; coloring substance; magnetic substance; or a metal particle such as gold colloid; a radioactive substance such as 125I, 131I, 32P, 3H, 35S, or 14C; a phosphorylated phenol derivative such as a nitrophenyl phosphate, luciferin derivative, or dioxetane derivative; or the like. The enzyme may be a dehydrogenase; an oxidoreductase such as a reductase or oxidase; a transferase that catalyzes the transfer of functional groups, such as an amino; carboxyl, methyl, acyl, or phosphate group; a hydrolase that may hydrolyzes a bond such as ester, glycoside, ether, or peptide bond; a lyases; an isomerase; or a ligase. The enzyme may also be conjugated to another enzyme. The enzyme may be detected by enzymatic cycling. For example, when the detectable label is an alkaline phosphatase, a measurements may be made by observing the fluorescence or luminescence generated from a suitable substrate, such as an umbelliferone derivative. The umbelliferone derivative may comprise 4-methyl-umbellipheryl phosphate. The fluorescent or chemiluminescent label may be a fluorescein isothiocyanate; a rhodamine derivative such as rhodamine β isothiocyanate or tetramethyl rhodamine isothiocyanate; a dancyl chloride (5-(dimethylamino)-1-naphtalenesulfonyl chloride); a dancyl fluoride; a fluorescamine (4-phenylspiro&Isqb;furan-2(3H); ly-(3yH)-isobenzofuran&rsqb:-3;3y-dione); a phycobiliprotein such as a phycocyanine or physoerythrin; an acridinium salt; a luminol compound such as lumiferin, luciferase, or aequorin; imidazoles; an oxalic acid ester; a chelate compound of rare earth elements such as europium (Eu), terbium (Tb) or samarium (Sm); or a coumarin derivative such as 7-amino-4-methylcoumarin. The label may also be a hapten, such as adamantine, fluoroscein isothiocyanate, or carbazole. The hapten may allow the formation of an aggregate when contacted with a multi-valent antibody or (strep)avidin containing moiety. Further examples of detectable moieties include, but are not limited to, the following: radioisotopes (e.g. 3H, 14C3 35S, 123I, 125I, 131I 99Tc, 111In, 90Y, 188Re), radionuclides (e.g. 11C, 18F, 64Cu), fluorescent labels (e.g. FITC, rhodamine, lanthanide phosphors, carbocyanine), enzymatic labels (e.g. horseradish peroxidase, β-galactosidase, luciferase, alkaline phosphatase), chemiluminescent, biotinyl groups and predetermined polypeptide epitopes recognised by a secondary binding entity (e.g. leucine zipper pair sequences, binding sites for secondary antibodies, metal binding domains, epitope or protein tags, carbohydrates). In some embodiments, labels are attached by spacer arms of various lengths to reduce potential steric hindrance.

In indirect labelling, an additional molecule or moiety is brought into contact with, or generated at the site of, the antibody-antigen complexes, i.e. immune-complexes, between the primary antibody and the protein it binds to. For example, a detectable moiety such as an enzyme can be attached to or associated with the detecting antibody or detecting molecule as exemplified herein. The signal-generating molecule can then generate a detectable signal at the site of the immune-complex. For example, an enzyme, when supplied with suitable substrate, can produce a visible or detectable product at the site of the immune-complex.

As another example of indirect labelling, an additional molecule (which can be referred to as a binding agent) that can bind to either the molecule of interest or to the antibody (primary antibody) of interest, such as a second antibody to the primary antibody, can be contacted with the immunocomplex. The additional molecule can have signal-generating molecule or detectable moiety.

The additional molecule may be an antibody, which can thus be termed a secondary, tertiary or further antibody. Binding of a secondary antibody to the primary antibody can form a so-called sandwich with the first (or primary) antibody and the molecule of interest. The immune-complexes can be contacted with the labelled, secondary antibody under conditions effective and for a period of time sufficient to allow the formation of secondary immune complexes. The secondary immune complexes can then be generally washed to remove any non-specifically bound labelled secondary antibodies, and the remaining label in the secondary immune complexes can then be detected. The additional molecule can also be or include one of a pair of molecules or moieties that can bind to each other, such as the biotin/avadin molecules, and the detecting antibody or detecting molecule should then include the other member of the pair.

Further examples of indirect labelling include the detection of primary antibody-antigen (immune-complexes) by a two step approach. For example, a molecule (which can be referred to as a first binding agent), such as an antibody, that has binding affinity for the primary immune complex between the primary antibody-antigen complex can be used to form secondary complexes, e.g. if a secondary antibody, secondary immune-complexes, as described above. After washing, the secondary complex can be contacted with another further molecule (which can be referred to as a second binding agent) that has binding affinity for the first binding agent, again under conditions effective and for a period of time sufficient to allow the formation of tertiary complexes, e.g. if antibody a tertiary immune-complex. In this example the second binding agent may be linked to a detectable moiety, allowing detection of the tertiary complexes thus formed. This system may further comprise means to provide for signal amplification.

Other examples of primary, secondary or further binding agents with means for signal amplification are conjugated anti-immunoglobulins such as biotinylated antibodies (e.g., conjugated with avidin/streptavidin) or staphylococcal Protein A (binds IgG), Protein G, dextran, aptamers, proteins, peptides, small organic molecules, natural compounds (e.g. steroids), non-peptide polymers, or any other molecules that specifically and efficiently bind to other molecules conjugated with a detectable moiety of not.

### Methods of Evaluating Tissue Pre-Treatment

Further aspects of the present invention relates to a method for evaluating tissue pre-treatment, i.e. pre-analytical parameters, comprising providing a formalin fixed biological sample and including in an immunohistochemical (IHC) process or assay an internal control comprising one or more antibody that demonstrates variations in accessibility or binding capacity relative to variations in tissue pre-treatment.

This, the invention provides a method for evaluating tissue pre-treatment in an immunohistochemical process, comprising
a) providing a formalin fixed biological sample,
b) providing an internal control comprising one or more antibody that demonstrates variations in antigen accessibility or variations in antibody binding capacity to said antigen relative to variations in tissue pre-treatment
c) detecting said variations in antigen accessibility or variations in antibody binding capacity to said antigen in step b), and
d) analyzing said variations detected in step c) relative to variations in tissue pre-treatment thereby evaluating tissue pre-treatment.

Further embodiments are wherein the variation in tissue pre-treatment comprises fixation time, ischemic time and alcohol time.

Further embodiments are wherein the variation in tissue pre-treatment is fixation time.

Further embodiments are wherein the variation in tissue pre-treatment is ischemic time.

Further embodiments are wherein the variation in tissue pre-treatment is alcohol time.

Embodiment of the invention relates to a method, comprising the steps:
providing a biological sample
performing an immunohistochemical process including an internal control antibody
determining if pre-analytical process was appropriate compared to the immunohistochemical process by visualizing the internal control antibody

One further embodiment of the invention relates to a method wherein the immunohistochemical process includes the steps of staining the sample with an antibody and detecting directly or indirectly of the antibody.

### Immunohistochemistry Assay

The tissue samples needs to be prepared in order to work in an IHC assay. The tissue samples needs to be cut in appropriate sections, such as e.g. about 4 μm or appropriate to fit the method. Normally the tissue is, as described herein, formalin-fixed, paraffin-embedded tissue sections. The IHC is normally done after a heat-induced epitope retrieval (HIER, Dako), or by treating the tissues using EnVision™ FLEX Target Retrieval Solution, High pH (10×), (Dako Autostainer/Autostainer Plus). Further examples of antigen retrieval is Water bath methods using conventional methods know in the art, water bath methods using DAKO PT Link (http://pri.dako.com/00091_demasking_antigens_us.pdf), pressure cocker heating, autoclave heating, microwave oven heating, proteolytic pre-treatment, combined proteolytic pre-treatment and HIER, combined deparaffinization and target retrieval.

One example of preparing de-parafinized sections is that sections may be deparaffinized by means to deparaffinize formalin-fixed, paraffin-embedded tissue sections, e.g. by using Dako PT Link (Dako). Following procedure for EnVision™ FLEX Target Retrieval Solution, High pH (10×), (Dako Autostainer/Autostainer Plus) (Code K8014) the following parameters should be used for PT Link: Pre-heat temperature: 65° C.; epitope retrieval temperature and time: 97° C. for 20 (±1) minutes; cool down to 65° C. Remove Autostainer slide rack with slides from the PT Link tank and immediately dip slides into a jar/tank (e.g., PT Link Rinse Station, Code PT109) containing diluted room temperature EnVision™ FLEX Wash Buffer (10×), (Dako Autostainer/Autostainer Plus) (Code K8010). Leave slides in Wash Buffer for 1-5 minutes.

For paraffin-embedded sections, an aqueous mounting medium for coverslipping may be used (Dako Faramount Code S3025). As alternative specimen preparation, both deparaffinization and epitope retrieval may be performed in the PT Link using a modified procedure. After the staining procedure has been completed, the sections may be air dried at 60° C., immersed in xylene and mounted using permanent mounting medium. Alcohol should be avoided with permanent mounting as it may diminish reactivity of the red choromogen.

Before mounting, the tissue sections should not dry out before or during the following immunohistochemical staining procedure.

The methods provided here in may be performed manually, or, preferably, on an automated staining device. Thus, in one embodiment the methods are performed manually.

In further embodiments, the methods are performed on an automated staining device.

In a further embodiment, the methods provided herein may be used in tissue micro arrays. Tissue micro arrays are also known and described in the art. Typically, tissue micro arrays may typically contain 50 to 500 tissues on a single slide.

Examples of automated staining devices useful according to the present invention are to include, but not limited to, Dako Autostainer (DakoCytomation), BioGenex 16000TH (Biogenex), Nemesis™ (BIOCARE), and NexES, Benchmark, Capilary gp stainer (Ventana systems). The sample is then ready for visualisation, detection, an optional scoring and further analysis.

Visualisation and detection may be performed by using reagents readily available in the art. Examples of useful detection and visualization reagents and systems are polymer detection systems such as EnVision™ DuoFLEX doublestain System, high pH, (DAKO).

Further embodiments are wherein the detection is made manually, such as by a pathologist or a medical doctor or anyone equally trained to manually view and detect proteins by immunological staining, such as immunohistochemical straining on prostate tissue.

In further embodiments, the detection is made by image analysis. Suitable image analysis devices useful according to the present invention are to include, but are not limited to ACIS® III (Dako).

### One or More Internal Control Antibodies

Embodiment of the invention relates to a method, comprising the steps:
providing a biological sample
performing an immunohistochemical process including an internal control antibody
determining if pre-analytical process was appropriate compared to the immunohistochemical process by visualizing the internal control antibody

Embodiment of the invention relates to a method wherein the immunohistochemical process includes the steps of staining the sample with one or more antibody and detecting directly or indirectly of the antibody. Said one or more antibody is sensitive to pre-analytical parameters and pre-analytical conditions affecting the accessibility of the protein or antigen to which it binds specifically or to the binding capacity of said one or more antibody. Said one or more antibody thus acts as an internal control antibody providing a signal when detected and analyzed dependent on the pre-treatment of the tissue.

Embodiment of the invention relates to a use of one or more antibodies binding specifically to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15 and CK19 as an internal control for pre-analytical variations prior in an immunohistochemical process.

Thus, further embodiments are wherein the one or more antibody that demonstrates variations in antigen accessibility or variations in antibody binding capacity to said antigen is selected from the list consisting of antibodies binding specifically to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15, CK19 and 34βE12.

Further embodiments are wherein the internal control comprises one or more antibodies binding specifically to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15 or CK19 as an internal control determining tissue pre-treatment variations in an immunohistochemical process.

One further embodiment is wherein the internal control comprises one or more antibodies binding specifically to Caldesmon.

One further embodiment is wherein the internal control comprises one or more antibodies binding specifically to Vimentin.

One further embodiment is wherein the internal control comprises one or more antibodies binding specifically to Desmin.

One further embodiment is wherein the internal control comprises one or more antibodies binding specifically to Collagen type IV.

One further embodiment is wherein the internal control comprises one or more antibodies binding specifically to CD20.

One further embodiment is wherein the internal control comprises one or more antibodies binding specifically to CD3.

One further embodiment is wherein the internal control comprises one or more antibodies binding specifically to ERα.

One further embodiment is wherein the internal control comprises one or more antibodies binding specifically to Mammaglobin.

Further embodiments are wherein the variation in tissue pre-treatment is fixation time and the one or more antibody that demonstrates variations in antigen accessibility or variations in antibody binding capacity to said antigen is selected from the list consisting of antibodies binding to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15, CK19 and 34βE12.

Further embodiments are wherein the variation in tissue pre-treatment is ischemic time and the one or more antibody that demonstrates variations in antigen accessibility or variations in antibody binding capacity to said antigen is selected from antibodies binding to phosphorylated antigens.

One particular embodiment is wherein the phosphorylated antigen is pAkt.

The invention is with special reference to formalin fixed paraffin embedded (short FFPE) tissues. Thus, one further embodiment is wherein the tissue is formalin fixed and paraffin embedded (FFPE).

Formalin fixation is the pre-treatment where the tissue is fixed by formalin. During the fixation process the tissue is affected. The fixation stops the degradation process at the time point of fixation and is thus dependent on the thickness of the tissue which influences the time of fixative, i.e. formalin, penetration to the center of the tissue. Examples of other fixation treatments are given herein.

Alcohol pre-treatment has two main effects on the tissue; dehydration and fixation of particular proteins. Thus, accordingly, during this pre-treatment the tissue is affected. The dehydration prepares the tissue for paraffin embedding, i.e. paraffin infiltration by removal of water.

A biological sample in the context of the present invention can be a histological or cytological sample. Especially the present invention relates to the processing of a thin biological sample, e.g. a tissue section.

A further embodiment is wherein the internal control is biomarker specific, i.e. comprises at least one antibody binding specifically to said biomarker.

ERα and Kappa are two examples of biomarkers disclosed herein.

A further embodiment is wherein the internal control is tissue specific i.e. comprises at least one antibody binding specifically to said tissue. Examples of tissue-markers are CD (cluster designation or cluster of differentiation)-markers and Melan-A/MART-1.

Further embodiments are wherein the internal control is specific for a tissue selected from the list consisting of stomach, small intestine, colon, liver, kidney, heart, lung, duodenum, tounge, pylorus, pancreas, uterus, skin, gal bladder, urinary bladder, adrenal, muscle, and ovary.

Further embodiments are wherein the tissue is human tissue.

The various steps and the results of traditional immunohistochemical (IHC) staining are dependent on the pre-treatment and its pre-analytical conditions and its steps. Example of steps are e.g. i) time from resection till immersion into fixative, i.e. the ischemic step defined by ischemic time, ii) fixation step, e.g. formalin fixation defined by fixation time and formalin concentration and fixation temperature, and iii) Alcohol fixation step, defined by alcohol fixation time, alcohol concentration. All steps i-iii above are further dependent on tissue size.

Different pre-analytical steps and pre-analytical parameters creates a variability in different pre-analytical conditions, such as conditions for ischemic time, fixation time and alcohol time. Antigen retrieval, which is not part of the pre-analytical but of the analytical steps in an IHC assay, needs for example to take much longer time if the biological sample has undergone a long fixation time. On the other hand, standard antigen retrieval may destroy both morphology and antigenicity of a sample that has not been sufficiently long in fixative. In trying to control these variations, guidelines for conducting the pre-analytical steps have been issued and it is usually assumed that these guidelines have been followed when performing the immunohistochemical staining process. However, if the guidelines have not been followed, the information retrieved from the subsequent IHC staining is possibly not correct and the IHC assay could therefore give wrong diagnostic results. Furthermore, it is not possible to visualize from the stained biological sample if the guidelines for pre-analytical processes have been followed due to the lack of controls relating to the pre-analytical conditions of the pre-treatment. Hence, there is a great danger that incorrect diagnostic results are not recognized as incorrect but are used as such with possible wrong patient treatment as result.

The present invention provides a method that demonstrates, either in a separate sample or within the same sample, if the results of the analytical stain can be interpreted as the true results. Thus, in one embodiment there are no variations in the pre-analytical parameters and thus the pre-analytical parameters of the pre-treatment will not influence the staining and its results as determined by the one or more control antibodies.

In further embodiments variations in the pre-analytical parameters of the pre-treatment does not influence the staining and its results as determined by the one or more control antibodies.

In still further embodiments variations in the pre-analytical parameters of the pre-treatment does influence the staining and its results as determined by one or more of the internal control antibodies.

Thus, the one or more control antibodies and its use in the method of the present invention allows to determine if the IHC assay and its results may be determined as true results or not.

The method provides that the interpretation of the results as true or not can be obtained by using one or more internal control antibodies binding specifically to certain proteins, antigens, which are known to show varying results if the pre-analytical parameters vary. The reliability of the stained sample can thus be determined by increased, decreased or no change in staining intensity of the control antibody or even presence or non-presence of the staining intensity of the control antibody. For example, if the biological sample has undergone a too long fixation step in the pre-treatment of the tissue sample the staining of the control antibody will show reduced staining intensity or not be visible at all in the tissue sample. Thus, the analytical biomarker that varies in its staining intensity in the same way as the one or more control antibodies is thus determined to have the same reduced staining intensity as its pre-determined one or more control antibodies. This will clearly indicate to the user the reliability of the staining and its results, applied in this particular case to be less reliant since reduced staining intensity.

Thus, one embodiment is wherein the variation in tissue pre-treatment is alcohol time and the one or more antibody that demonstrates variations in antigen accessibility or variations in antibody binding capacity to said antigen is antibodies binding to estrogen receptor (ER). Further embodiments are wherein the method further comprises determining if said tissue pre-treatment is acceptable based on the detected and analyzed variations.

Traditional steps from start of fixation in the pre-treatment steps up to but not including the analytical immunohistochemical staining are; formalin fixation, immersion into alcohol 70%, alcohol 95%, alcohol 100%, and further alcohol 100%, xylene, paraffin embedding.

In further embodiments, some of the steps can be applied multiple times, for example to wash away the previous step the first time and then have full action in the subsequent rounds of the same step when repeated multiple times. Multiple times may be repeated 1×, 2×, 3×, 4×, 5×, 6×, 7×, 8×, 9×, or even 10× or more times.

The staining with the control antibody in a biological sample may in one embodiment be conducted by replacing the analytical antibody staining an analytical biomarker with the control antibody in a parallel biological sample in the immunohistochemical process. The result will in this embodiment indicate variations in the pre-analytical parameters in the pre-treatment of the tissue sample in a separate parallel sample of the sample where analysis of an analytical biomarker is being performed. Said parallel control sample can be used for different analytical biological samples as long as they all have undergone the same tissue pre-treatment steps and the same subsequent analytical immunohistochemical assay.

The one or more control antibody may in further embodiments also be included in the same biological sample in the analytical immuohistochemical process (assay) either i) together with the analyzing analytical antibody binding to the analytical biomarker or ii) included (added) sequentially to the analyzing antibody.

In an embodiment wherein the one or more control antibody is added together with the analyzing analytical antibody, i.e. i) above, the one or more control antibody may be mixed with the analytical antibody before adding to the biological sample or be available in a pre-mixed solution either in a concentrated form to be diluted upon use to a working concentration or in a ready-to-use concentration.

In an embodiment wherein the one or more control antibody is added sequentially to the analyzing antibody, the one or more control antibody may be added before or after the analyzing antibody in time and in separate steps. E.g. the analyzing antibody is added to the biological sample and subsequently incubated with the sample. The sample is then washed and the next antibody, then the one or more control antibody is added. Further embodiments are wherein the opposite, i.e. adding of one or more control antibody to the biological sample, incubating and washing before the analyzing antibody is done.

The IHC analytical process (assay) where the control antibody is applied simultaneously with the analyzing antibody may include the steps:

a) target retrieval
b) wash
c) adding of primary antibody and at least one internal control antibody
d) co-incubation of antibodies
e) wash
f) adding of detection system
g) wash
h) adding of chromogen
i) wash
j) mounting with coverslip or other means to protect, and
k) analysis of the staining manually or by automatic means.

The IHC analytical process (assay) where the control antibody is not applied simultaneously but sequentially with the analyzing antibody may include the steps:

a) target retrieval
b) wash
c) adding of primary analyzing antibody
d) wash
e) adding of at least one internal control antibody
f) wash
g) adding of detection system
h) wash
i) adding of chromogen
j) wash
k) mounting with coverslip or other means to protect, and
l) analysis of the staining manually or by automatic means.

Step c) and e) in the embodiment wherein the control antibody is not applied simultaneously but sequentially with the analyzing antibody may be equally changed in order, meaning that addition of the one or more internal control antibody may precede addition of the analytical antibody.

In further embodiments, one or more analyzing antibodies are added. An analyzing antibody is sometimes referred to as a clinical antibody.

In further embodiments, the detection system may be done separately and sequentially for each antibody added, e.g. analyzing antibody or the at least one internal control antibody. This is particularly relevant for embodiments where the analyzing antibody and the at least one internal control antibody (e.g. both the analyzing antibody and the at least one antibody are considered to be the primary antibodies) is of the same species, e.g. both are murine or both are rabbit antibodies. Then, each antibody needs to be detected separately and before the next primary antibody is added to the tissue sample.

In some embodiments the detection system may be done simultaneously for all the antibodies added, e.g. analyzing antibody or the at least one internal control antibody. This is may be in embodiments where the analyzing antibody and the at least one internal control antibody (e.g. both the analyzing antibody and the at least one antibody are considered to be the primary antibodies) is of different species, e.g. one of the antibodies is murine and the other one is Swine or rabbit or sheep antibody. If this is the case, the primary antibodies i.e. the analyzing antibody or the at least one internal control antibody, may be detected together. However, this does not exclude the possibility that the primary antibodies, despite them being of different species, are detected separately, i.e. sequentially, as given above for primary antibodies of the same species.

Processes using secondary or further antibodies are also enclosed.

More than one control antibody can be used in the staining of a single sample, hence, giving the user the possibility to verify if all the pre-analytical parameters in the tissue pre-treatment have been upheld during the staining and to verify if, and to what extent, any deviations giving rise to variations in the pre-analytical parameters in the tissue pre-treatment have been made.

In one embodiment it is also possible to select the verification of one or more pre-analytical parameter of the tissue pre-treatment, by including respectively one or more control antibody in the staining, where each control antibody verifies a separate parameter such as e.g. one internal control antibody is an internal control of ischemic time, one internal control antibody is an internal control of fixation time, one internal control antibody is an internal control of potential over fixation and one internal control antibody is an internal control of under fixation of the tissue.

For example, an antibody binding specifically to Caldesmon, Vimentin, Desmin, or Collagen type IV may be an internal control antibody for over fixation, i.e. a too long fixation, of a tissue, either in a tissue specific manner or in a biomarker specific manner, wherein the fixation is formalin.

Further, an antibody binding specifically to CD20, CD3, ERα, or Mammaglobin may be an internal control for an under fixation, i.e. fixation too short time, of a tissue either in a tissue specific manner or in a biomarker specific manner, wherein the fixation is an alcohol, such as methanol or ethanol.

In one embodiment, one control antibody only may be used to indicate if many pre-analytical parameters during tissue pre-treatment have been upheld. One control antibody may in such a way indicate if both fixation time versus the immunohistochemical analytical process (assay) and the ischemic time versus the immunohistochemical analytical process (assay) are acceptable, although the one control antibody might not be able to discriminate which of the two factors, i.e. pre-analytical parameters, that varies or lack if the control antibody is not within the accepted staining intensity.

The control antibody may be visualized with visualization systems known in the art, such as fluorescent labels, luminescent labels, chromogens etc. or other visualization systems described herein.

The control antibody may also be differentiated and distinguished from the analytical antibody by providing a specific diagnostic stain, due to specific localization of the one or more control antibody in the tissue tomography area and being different from the location of specific diagnostic stain of the analytical antibody.

Antibodies to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15 and CK19 are examples of antibodies suitable as internal control for the pre-analytical treatment of the tissue pre-treatment.

Antibodies to CD3, S100, CD31, CD20, EMA and CK19 are particularly suitable as internal control for formaling fixation time.

Antibodies to CD3, for example, shows variations in signal when formalin fixation has been either too short or too long. CD3 also shows variations in signal when the ischemic time has been too long.

Antibodies raised against phosphorylated antigens, for example pAkt, are particularly suitable as internal controls for determining if the ischemic time is acceptable and to determine if variations thereof for the subsequent immunohistochemical process. Examples of other phosphorylated antigens are pHER2, pEGFR, pS6, p70 S6 kinase, pERK1/2 (also called pMAPK), pIGF1R, p44/42 MAPK, pGSK3b(ser9), and pERK.

For alcohol time an ER antibody is suitable as an internal control antibody for determining if the alcohol time is acceptable and to determine if variations thereof for the subsequent immunohistochemical process.

### Guidance for Corrective Measures

Depending on the staining intensity in the subsequent staining analysis of the internal control antibody and the specific analyzing antibody, guidance can be given for possible corrective measures.

Examples of how guidance can be given are:
The internal control signal is outside the acceptable e.g. by example a signal intensity scored as 2.5+/−0.25 wherefore the specific signal cannot be trusted due to the +/−0.25 variability and the IHC assay analytical test shall be repeated. This could for example be the case if one internal control antibody was used for determining the suitability of more than one pre-analytical parameters.
The internal control signal is outside the acceptable (by example 2.5+/−0.25) wherefore the specific signal cannot be trusted due to the +/−0.25 variability and the test shall be repeated with increased target retrieval time from 20 minutes to 40 minutes at maximal temperature. This is for example the case if the formalin fixation time has not been appropriate, for example too long, in relation to the target retrieval process in the subsequent immunohistochemical process.
The internal control signal is outside the acceptable (by example 2.5+/−0.25) wherefore the specific signal cannot be trusted due to the +/−0.25 variability and the test shall be repeated with longer specific incubation time of the antibody and internal control antibody, by example 40 minutes instead of 20 minutes.
The internal control antibody signal is outside the acceptable (by example 2.5+/−0.25) wherefore the specific signal cannot be trusted due to the +/−0.25 variability and the test shall be repeated with longer detection system incubation time, by example 40 minutes instead of 20 minutes.
The internal control signal is outside the acceptable (by example 2.5+/−0.25) wherefore the specific signal cannot be trusted due to the +/−0.25 variability and the test shall be repeated with longer chromogen incubation time, by example 15 minutes instead of 10 minutes.
The internal control signal is outside the acceptable (by example 2.5+/−0.25) wherefore the specific signal cannot be trusted as it is due to the +/−0.25 variability, but by using a specific analytical antibody signal related table or algorithm, the correct specific analytical antibody signal can be calculated. For example a 0.75 grades decreased internal control antibody signal will indicate a specific antibody correction of +0.5 grade.

The visualization can suitably be performed manually by the user or by using image analysis systems known in the art.

### Uses of the Composition

Further aspects of the present invention include uses of the methods and internal controls as provided herein.

One further aspect of the present invention provides use of one or more internal control for determining tissue pre-treatment variations in an immunohistochemical process.

Further embodiments are wherein the internal control comprises one or more antibodies binding specifically to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15 and CK19 as an internal control determining tissue pre-treatment variations in an immunohistochemical process.

Even further embodiments are wherein the internal control is tissue specific.

Even further embodiments are wherein the internal control is biomarker specific.

### Kits

The present invention also provides kits for immunoassays such as immunohistochemistry comprising at least one or more internal control antibody.

Thus, a further aspect of the present invention provides a kit for evaluating tissue pre-treatment comprising at least one or more internal control antibody, and instructions to perform the method according to the present invention.

Further embodiments are wherein the kit further comprises at least one analyzing antibody.

Further embodiments are wherein the kit further comprises guidance of interpretation of tissue pre-treatment variation(s).

Further embodiments are wherein the kit comprises at least one or more analytical, or analyzing, antibodies.

Still a further embodiment provides a kit for immunoassays comprising a) at least one or more internal control antibody, b) at least one analytical antibody, and c) optionally, instructions for using said internal controls.

Further embodiments include visualisation reagents to be able to detect the antibodies in the kit.

Examples of visualisation and detection reagents are known in the art and given in e.g. Antibodies: A Laboratory Manual, Harlow and Lane, (Cold Spring Harbor Laboratory press, Cold Spring Harbor, N.Y. 1988), Current Protocols in Immunology, (Unit 21.4, 2003) and Current Protocols in Molecular Biology, (Unit 14.6, 2001, the latter two of John Wiley and Sons, Inc., N.Y.).

In some kit embodiments, the primary antibody can be directly labelled as described herein. Other kit embodiments will include secondary or further detection such as secondary antibodies (e.g., goat anti-rabbit antibodies, rabbit anti-mouse antibodies, anti-hapten antibodies) or non-antibody hapten-binding molecules (e.g., avidin or streptavidin) as described herein. In such kits, the secondary or further detection means may be directly labelled with a detectable moiety. In other instances, the secondary (or further) antibody or binding agent will be conjugated to a hapten (such as biotin, DNP, and/or FITC), which is detectable by a detectably labelled cognate hapten binding molecule (e.g.; streptavidin (SA) horseradish peroxidase, SA alkaline phosphatase). Some kit embodiments may include colorimetric reagents (e.g., DAB, and/or AEC) in suitable containers to be used in concert with primary or secondary (or higher order) detection means (e.g., antibodies or binding entities) that are labelled with enzymes for the development of such colorimetric reagents.

In some embodiments, a kit includes positive or negative control samples, such as a cell line or tissue known to express or not express a particular protein or antigen.

In some embodiments, a kit includes instructional materials disclosing, for example, means of use of the methods disclosed herein, means of use of the at least one or more internal controls, guidance of its interpretation e.g. such but not limited to as disclosed herein, and means of use of one or more analysing antibody or means for use of a particular reagent. The instructional materials may be written, in an electronic form (e.g., computer diskette or compact disk) or may be visual (e.g., video files).

The kits may also include additional components to facilitate the particular application for which the kit is designed. Thus, for example, the kit can include buffers and other reagents routinely used for the practice of a particular disclosed method. Such kits and appropriate contents are well known to those of skill in the art.

The kit may further comprise, in an amount sufficient for at least one assay, the composition according to the invention as a separately packaged reagent.

Instructions for use of the packaged reagent are also typically included. Such instructions typically include a tangible expression describing reagent concentrations and/or at least one assay method parameter such as the relative amounts of reagent and sample to be mixed, maintenance time periods for reagent/sample admixtures, temperature, buffer conditions and the like.

Certain kit embodiments can include a carrier means, such as a box, a bag, a satchel, plastic carton (such as moulded plastic or other clear packaging), wrapper (such as, a sealed or sealable plastic, paper, or metallic wrapper), or other container.

In some examples, kit components will be enclosed in a single packaging unit, such as a box or other container, which packaging unit may have compartments into which one or more components of the kit can be placed. In other examples, a kit includes a one or more containers, for instance vials, tubes, and the like that can retain, for example, one or more biological samples to be tested.

Other kit embodiments include, for instance, syringes, cotton swabs, or latex gloves, which may be useful for handling, collecting and/or processing a biological sample. Kits may also optionally contain implements useful for moving a biological sample from one location to another, including, for example, droppers, syringes, and the like. Still other kit embodiments may include disposal means for discarding used or no longer needed items (such as subject samples, etc.). Such disposal means can include, without limitation, containers that are capable of containing leakage from discarded materials, such as plastic, metal or other impermeable bags, boxes or containers.

Non-limiting examples which embody certain aspects of the invention will now be described.

### EXAMPLES

### Example 1

To demonstrate formalin fixation time variations in immunohistochemical signal, all tissues were prior to formalin exposure divided into 5 parts that were each exposed to different formalin fixation duration.

Tissue samples: tonsil (benign), skin (melanoma), breast (adenocarcinoma).

Tissues were frozen at −80° C., sectioned at 1×1×0.2 cm in size, and then fixed at 1, 4, 24, 48, and 120 hours in 10% NBF before the ethanol dehydration step and paraffin infiltration in a tissue processor (Shandon Tissue Processor).

### Reagents/Instruments:

### A.) Peroxidase Blocking Reagent (PBR)-(DM801)

### B.) Horseradish Peroxidase (HRP)-(DM802)

### C.) DAB+-(DM807)

### D.) Hematoxylin-(S3301)

E.) TBST buffer (blueing reagent)
F.) Substrate buffer (FLEX)-(DM803)

### G.) ETOH (100% & 95%)

### H.) Xylene

I.) Antibody diluent-(S0809)

### G.) PT Module

### K.) Dako Autostainer Plus

| Step | Incubation Time |
|---|---|
| Pretreatment (Manual) | 50 minutes |
| Endogenous Peroxidase Block | 5 minutes |
| Primary Antibody | 20 minutes |
| Secondary Antibody with Enzyme (HRP) | 20 minutes |
| Substrate: DAB+ | 10 minutes |
| Counterstain: Hematoxylin | 5 minutes |

After completion of fixation and paraffin infiltration of tissues pieces, all pieces from one original tissue were placed in one multiblock for later sectioning and testing, thereby securing identical analytical treatment. All antibodies were tested on an Autostainer+ using the respective RTU antibody product program.

Tables A-D show antibodies showing changes in immunoreactivity due to fixation. Tables E-F show antibodies showing no changes in immunoreactivity due to fixation

TABLE A
Antibody: CD3

| | | | | Staining | |
|---|---|---|---|---|---|
| Hours | | | Tissue | Signal/ | |
| Fixated | Tissue | Dilution | Block | Intensity | Date |
| 1 | Tonsil (benign) | 10x | 37424 | 3.75 | Apr. 15, 2008 |
| 4 | Tonsil (benign) | 10x | 37424 | 3.5 | |
| 24 | Tonsil (benign) | 10x | 37424 | 3.5 | |
| 48 | Tonsil (benign) | 10x | 37424 | 3.25 | |
| 120 | Tonsil (benign) | 10x | 37424 | 3 | |

TABLE B
Antibody: 34βE12

| | | | | Staining | |
|---|---|---|---|---|---|
| Hours | | | Tissue | Signal/ | |
| Fixated | Tissue | Dilution | Block | Intensity | Date |
| 1 | Tonsil (benign) | 5x | 37424 | 4 | Apr. 15, 2008 |
| 4 | Tonsil (benign) | 5x | 37424 | 3.75 | |
| 24 | Tonsil (benign) | 5x | 37424 | 3.75 | |
| 48 | Tonsil (benign) | 5x | 37424 | 2.5 | |
| 120 | Tonsil (benign) | 5x | 37424 | 3.5 | |

TABLE C
Antibody: S100

| Hours | | | | Staining | |
|---|---|---|---|---|---|
| Fixated | Tissue | Dilution | Tissue Block | Signal/Intensity | Date |
| 1 | Melanoma | 4x | 37420 | 3.5 | |
| 4 | Melanoma | 4x | 37420 | 2 | |
| 24 | Melanoma | 4x | 37420 | 2 | |
| 48 | Melanoma | 4x | 37420 | 2 | |
| 120 | Melanoma | 4x | 37420 | 2 | |

TABLE D
Antibody: CD99

| Hours | | | | Staining | |
|---|---|---|---|---|---|
| Fixated | Tissue | Dilution | Tissue Block | Signal/Intensity | Date |
| 1 | Tonsil | RTU | 37425 | 3 | Jul. 10, 2008 |
| 4 | Tonsil | RTU | 37425 | 2.75 | |
| 24 | Tonsil | RTU | 37425 | 2.75 | |
| 48 | Tonsil | RTU | 37425 | 2.25 | |
| 120 | Tonsil | RTU | 37425 | 2.25 | |

TABLE E
Antibody: Progesterone Receptor (PR)

| | | | | Staining | |
|---|---|---|---|---|---|
| Hours | | | Tissue | Signal/ | |
| Fixated | Tissue | Dilution | Block | Intensity | Date |
| 1 | Breast Cancer | RTU | 37416 | 3 | Jul. 29, 2008 |
| 4 | Breast Cancer | RTU | 37416 | 3 | |
| 24 | Breast Cancer | RTU | 37416 | 3 | |
| 48 | Breast Cancer | RTU | 37416 | 3 | |
| 120 | Breast Cancer | RTU | 37416 | 3 | |

TABLE F
Antibody: IgM

| Hours | | | | Staining | |
|---|---|---|---|---|---|
| Fixated | Tissue | Dilution | Tissue Block | Signal/Intensity | Date |
| 1 | Tonsil | 16k | 37424 | 2.5 | |
| 4 | Tonsil | 16k | 37424 | 2.5 | |
| 24 | Tonsil | 16k | 37424 | 2.5 | |
| 48 | Tonsil | 16k | 37424 | 2.5 | |
| 120 | Tonsil | 16k | 37424 | 2.5 | |

0-4 Scale

| SCORE | STAINING INTENSITY |
|---|---|
| 0 | No Staining |
| 0.5 | Very weak staining |
| 1.0 | Weak staining |
| 1.5 | Weak to moderate staining |
| 2.0 | Moderate staining |
| 2.5 | Moderate to strong staining |
| 3.0 | Strong staining |
| 3.5 | Very strong staining |
| 4.0 | Extremely strong staining |

### Example 2

In the tissue processing the next step after formalin fixation is alcohol dehydration of the tissue. Whereas the alcohol removed water from the tissue through the use of increasing percentages of alcohol (often from 70% to absolute or 100%), alcohol in itself will also have some fixing effect. If the tissues have been properly fixed in formalin this will have no effect, but if not a not-short alcohol incubation time may give sufficiently additional fixation to make the tissue good for IHC testing.

Estrogen Receptor specific Ab is tested on tissue pieces that all originate from one patient sample, but are treated differently.

Three treatments are tested and immunohistochemical results compared. The immunohistochemical process is the same as in example 1:
a) Formalin for 12 hours followed by alcohol for 4 hours (1 hour incubation of each of 3 different graded alcohols followed by absolute alcohol).
b) Formalin for 1 hour followed by alcohol for 40 minutes (10 minutes incubation of each of 3 different graded alcohols followed by absolute alcohol).
c) Formalin for 1 hour followed by alcohol for 4 hours (1 hour incubation of each of 3 different graded alcohols followed by absolute alcohol).

a) and c) both give sufficient signal whereas b) provides underfixed tissue that displays decreased immunohistochemical signal.

1. A method for evaluating tissue pre-treatment in an immunohistochemical process, comprising
a) providing a formalin fixed biological sample,
b) providing an internal control comprising one or more antibody that demonstrates variations in antigen accessibility or variations in antibody binding capacity to said antigen relative to variations in tissue pre-treatment
c) detecting said variations in antigen accessibility or variations in antibody binding capacity to said antigen in step b), and
d) analyzing said variations detected in step c) relative to variations in tissue pre-treatment thereby evaluating tissue pre-treatment.

2. The method according to claim 1 wherein the variation in tissue pre-treatment comprises fixation time, ischemic time and alcohol time.

3. The method according to claim 1 wherein the variation in tissue pre-treatment is fixation time.

4. The method according to claim 1 wherein the variation in tissue pre-treatment is ischemic time.

5. The method according to claim 1 wherein the variation in tissue pre-treatment is alcohol time.

6. The method according to claim 1, wherein the one or more antibody that demonstrates variations in antigen accessibility or variations in antibody binding capacity to said antigen is selected from the list consisting of antibodies binding specifically to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15, CK19 and 34βE12.

7. The method according to claim 1, wherein the variation in tissue pre-treatment is fixation time and the one or more antibody that demonstrates variations in antigen accessibility or variations in antibody binding capacity to said antigen is selected from the list consisting of antibodies binding to CD3, S100, Melan A, Villin, ER α, CD20, EMA, E-Cadherin, CD9, Vimentin, IgG, Kappa, Myeloperoxidase, CD18, 34βE12, Chromogranin A, Mammaglobin, CD31, Caldesmon, CD15, CK19 and 34βE12.

8. The method according to claim 1, wherein the variation in tissue pre-treatment is ischemic time and the one or more antibody that demonstrates variations in antigen accessibility or variations in antibody binding capacity to said antigen is selected from antibodies binding to phosphorylated antigens.

9. The method according to claim 8, wherein the phosphorylated antigen is pAkt.

10. The method according to claim 1, wherein the variation in tissue pre-treatment is alcohol time and the one or more antibody that demonstrates variations in antigen accessibility or variations in antibody binding capacity to said antigen is antibodies binding to ERα.

11. The method according to claim 1, further comprising determining if said tissue pre-treatment is acceptable based on the detected and analyzed variations.

12. The method according to claim 1, wherein the internal control is tissue specific.

13. The method according to claim 1, wherein the internal control is an antibody binding specifically to a tissue selected from the list consisting of stomach, small intestine, colon, liver, kidney, heart, lung, duodenum, tongue, pylorus, pancreas, uterus, skin, gal bladder, urinary bladder, adrenal, muscle, and ovary.

14. The method according to claim 1, wherein the tissue is human tissue.

15. The method according to claim 1, wherein the internal control is biomarker specific.

16. The method according to claim 15, wherein the biomarker specific internal control is an antibody binding specifically to ERα or to Kappa.

17-20. (canceled)

21. A kit for evaluating tissue pre-treatment comprising at least one or more internal control antibody, instructions to perform the method according to claim 1.

22. The kit according to claim 21 further comprising at least one analyzing antibody.

23. The kit according to claim 21, further comprising guidance of interpretation of tissue pre-treatment variation(s).
